(19) United States
(12) Patent Application Publication
(10) Pub. No.: US 2021/0390399 A1
(43) Pub. Date: Dec. 16, 2021

(54) Predicting Physiological Parameters

(71) Applicant: IMPERIAL COLLEGE OF SCIENCE, TECHNOLOGY & MEDICINE, London (GB)

(72) Inventors: Pantelis GEORGIOU, London (GB); Pau HERRERO-VINAS, London (GB); Kezh LI, London (GB)

(73) Assignee: Imperial College of Science, Technology & Medicine, London (GB)

(21) Appl. No.: 17/290,850

(22) PCT Filed: Nov. 1, 2019
(86) PCT No.: PCT/GB2019/053115

§ 371 Date: May 3, 2021

(30) Foreign Application Priority Data
Nov. 1, 2018 (GB) 1817893.9

(51) Int. Cl. G06N 3/08; G06N 3/04

(52) U.S. Cl. CPC G06N 3/08; G06N 3/04

(57) ABSTRACT

The present disclosure relates to the prediction of physiological parameters, such as glucose levels. A training input is received, the training input comprising time-series values of a physiological parameter and time-series values of one or more further parameters. Training examples are generated form the training input, wherein each training example comprises a training dataset and a corresponding training label, wherein each training dataset is generated from the training input with time-series values restricted to a time interval, and wherein each corresponding training label represents the value of the physiological parameter a prediction period after the end of that time interval. A neural network is then trained using the training examples.

### FIELD

The present disclosure relates to the prediction of physiological parameters. In particular, the present disclosure relates to the prediction of blood glucose levels using neural network techniques.

### BACKGROUND

Predicting the evolution of a patient's health has long been of vital interest in the treatment of medical conditions because negative outcomes, if foreseen, can often be avoided by preventive treatment. Traditionally, this prediction has been performed by medical staff, based on observations on the patient and their theoretical and empirical understanding of the workings of the body.

A patient's health state can be quantitatively described by physiological parameters, such as concentrations of substances in the blood and in tissues, pressures of fluids, heart rate, breathing rate, and combinations of those parameters, among others. Negative health outcomes are often tied to a physiological parameter leaving a normal range.

In recent years, devices capable of continuously monitoring physiological parameters have multiplied, such that patients now have access to a wealth of present and historical data concerning their physiological parameters. One example is in the field of blood glucose measurements for patients suffering from diabetes. For example, portable continuous glucose monitoring (CGM) devices have recently enabled patients with Type 1 diabetes to continuously measure their blood glucose concentration.

While these approaches have improved the ability of patients to understand the current status of a given physiological parameter, such as blood glucose concentration, and therefore to take action at the point where such a parameter exceeds its usual bounds, there is an ongoing need to improve the ability to predict future changes. For example, it would be desirable for patients to predict in advance an oncoming hypo- or hyper-glycaemic episode, and thus to take action to ameliorate or avoid the consequences.

However, making such predictions is a difficult task, because the complexity of the physiological system. It is a continuing challenge to create adequate models to identify potential risk scenarios for adverse events, and as yet there is limited practical implementation of such models available for day-to-day patient use. While some proposals have suggested machine learning techniques may be able to identify patterns in the development of a physiological parameter over time, there remain significant hurdles to introducing these in practice.

### SUMMARY

According to a first aspect of the present disclosure, there is provided a method to train a neural network to predict a value of a physiological parameter. Namely, the method receives a training input comprising time-series values of a physiological parameter and time-series values of one or more further parameters. The method then generates one or more training examples from the training input: each training example comprises a training dataset and a corresponding training label. In each training example, the training dataset is generated from the training input where the time-series values used are restricted to a time interval specific to that training example. The corresponding training label represents the value of the physiological parameter a prediction period after the end of that time interval. The method then trains a neural network using the training examples, so that the trained neural network is able to generate a prediction label from a prediction dataset, where the prediction dataset is generated from a prediction input comprising time-series values of the physiological parameter and time-series values of one or more further parameters, and the prediction label represents the value of the physiological parameter a prediction period after the latest time-series value in the prediction dataset.

Using a neural network to predict the value of the physiological parameter enables the method to learn from the training examples to refine its understanding of the evolution of the physiological parameter given the training examples. Furthermore, by using the information comprised in the time-series values of the one or more further parameters in addition to the time-series values of the physiological parameter, the neural network is able to more accurately predict the future value of the physiological parameter, when the evolution of the physiological parameter is linked to the one or more further parameters. Finally, because every training label represents the value of the physiological parameter a prediction period after the end of the last value in the corresponding training dataset, all the examples train the neural network on the same task, that of predicting the physiological parameter a prediction period in the future. In this way, given enough examples, the neural network becomes suited to this task, and so is able to carry out the task on the prediction dataset.

In some embodiments, generating the training dataset includes modelling time-series values of additional parameters based on the training input. In this way, meaningful information may be extracted from the training input before it is presented to the neural network for training, so that the content of the training datasets is enriched. In particular, in some embodiments, one or more of the additional parameters may represent modelled physiological parameters, estimated from the training input using a physiological model. This enables the known evolutions and interactions of the physiological parameter and the one or more further parameters to be incorporated into the datasets presented to the neural network, thereby reducing the complexity of the functional representation that the neural network itself needs to learn.

In some embodiments, the training label may take one of a given sets of values, so that the task of the neural network is simplified to predicting one of several categories. In particular, the training label may be the quantized change in the physiological parameter in the prediction period (i.e. a predetermined period) from the end of the time interval. This may take advantage of properties of the physiological parameter such as differentiability and invariance to its current value. Indeed if the physiological parameter is differentiable, changes in a small prediction period will be small, so that the range of possible values that the change in the physiological parameter can take is smaller than the range of the physiological parameter itself. In this way, the prediction of the physiological parameter may be represented with greater precision for a given number of categories. Furthermore, if the physiological parameter's evolution depends much less on its present value than on the further parameters, the change in the physiological parameter given the further parameters will be relatively identical whatever the physiological parameter's present value. By predicting a change of the physiological parameter, this assumption is encoded in the datasets, so that the neural network does not need to learn it.

Alternatively, the training label may be any function of the physiological parameter and the dataset which is invertible given the dataset; for example, the training label may be the real-valued or quantised physiological parameter after a predetermined period from the end of the time interval, or the real-valued change in the physiological parameter in the predetermined period from the end of the time interval.

In some embodiments, the neural network may be a convolutional neural network (CNN), so that each layer of the convolutional neural network extracts features of the datasets at different time-scales, with the shallower layers extracting short-term features and deeper layers combining the outputs of shallower layers into longer-term features. In this way, features of the datasets relevant to the prediction of the physiological parameter are extracted in an efficient way. In particular, the convolutional neural network may be a causal convolutional neural network, where one or more of the convolutional layers implement causal convolutions. Additionally or alternatively, the convolutional neural network may be a dilated convolutional neural network, where one or more of the convolutional layers implement dilated convolutions. Dilated convolutions increase the receptive field of the convolutional layers without increasing the number of parameters of the neural network, and therefore improve the merging of information between values in the dataset spread across in time.

Although some embodiments utilise dilated neural networks, alternative neural networks, optionally dilated neural networks, may be additionally or alternatively adopted. For example, the neural network may be a recurrent neural network (RNN) adapted to time-series prediction, such as a dilated RNN, a dilated Long Short-Term Memory (LSTM) or a dilated Gated Recurrent Unit (GRU).

In some embodiments, at least one of the output layers of the neural network may be directly connected using skip connections to another layer. For example, in a convolutional neural network, one of the output layers may be connected to a convolutional layer other than the deepest convolutional layer. In a recurrent neural network, one of the output layers may be connected to one of the recurrent hidden layers other than the deepest recurrent hidden layer. In this way, the particular output layer connected featuring skip connections is able to better merge the features extracted by the different layers, which corresponded to different time-scales.

Optionally, both the training input and the prediction input may further comprise a value for each of one or more time-invariant parameters, one or more of which may represent factors varying between individuals which influence the evolution of the physiological parameter. In such an embodiment, one or more of the layers of the neural network may additionally operate as a function of one or more of the one or more time-invariant parameters. By taking into account the dependencies of the physiological parameter on the time-invariant parameters, the neural network is rendered flexible to variation between individuals, so that training the neural network on data taken from multiple individuals is possible without losing accuracy. As a result, the neural network may be pre-trained on data taken from a population of individuals, such that if the trained network is applied to a new patient, the prediction will take into account the individual characteristics of the new patient, thereby improving the accuracy of predictions and reducing the amount of data sourced from the new patient that is required to train the network to a satisfactory prediction accuracy.

In some embodiments, one or more of the neural network's layers may include a gated activation function. This improves the flexibility of the network to represent a highly non-linear mapping.

In some embodiments, generating the training examples may include removing outlier values and interpolating missing values in the time-series values of the training input. This improves the quality and quantity of the data used for training.

In some embodiments, one or more of the further parameters in the training input may represent the occurrence of lifestyle events.

The method may also comprise using the neural network to predict a future value of the physiological parameter, for example by generating a prediction label from a prediction input. This may be based on one or more measurements of the physiological parameter. For example, there is also provided a method consisting in generating a prediction label from a prediction input, using the neural network trained by a method according to one of the preceding embodiments. Additionally, the generated prediction label may be used to control the automatic operation of a device configured to inject a therapeutic substance in a patient. Alternatively or additionally, the prediction label may be displayed to a user.

There is also provided a data processing system comprising one or more processors adapted to perform one or more of the methods of the present disclosure.

There is also provided a computer program product comprising instructions which, when executed by a computer, cause the computer to carry out one or more of the methods of the present disclosure.

There is also provided computer-readable medium comprising instructions which, when executed by a computer, cause the computer to carry out one of more of the methods of the present disclosure.

### BRIEF DESCRIPTION OF THE FIGS.

Preferred embodiments of the present disclosure are described below with reference to the accompanying drawings, in which:

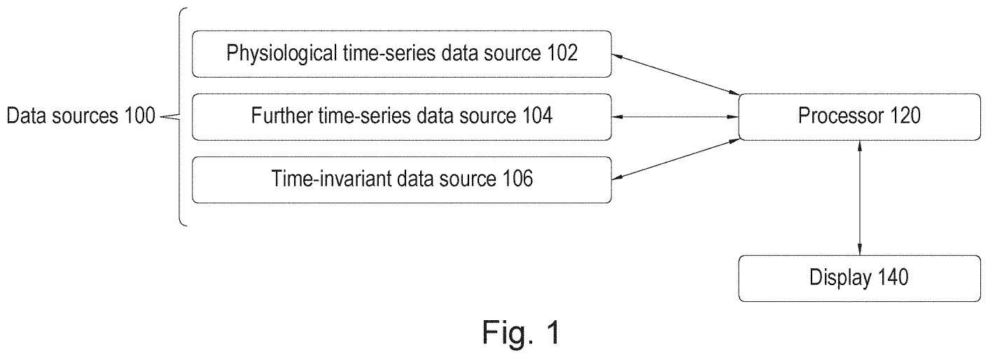

FIG. 1 illustrates an example system for the prediction of a physiological parameter;

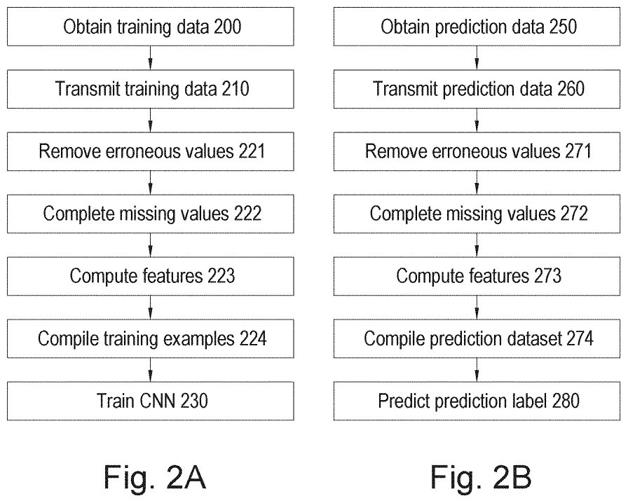

FIG. 2 shows an exemplary process for training and operating the neural network implemented by the system of FIG. 1;

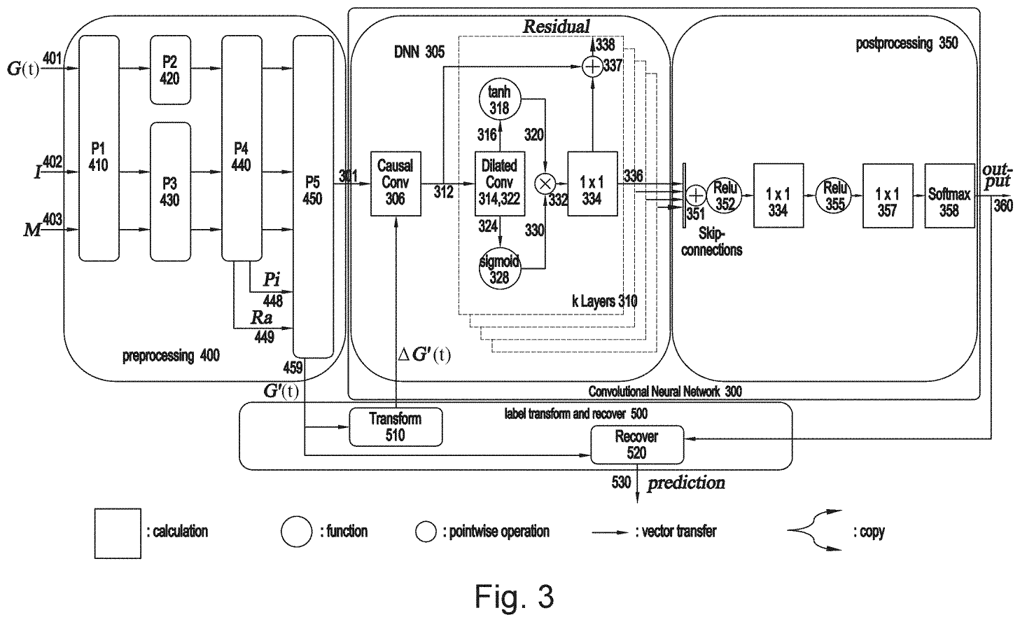

FIG. 3 shows the further detail of the neural network together with associated pre-processing;

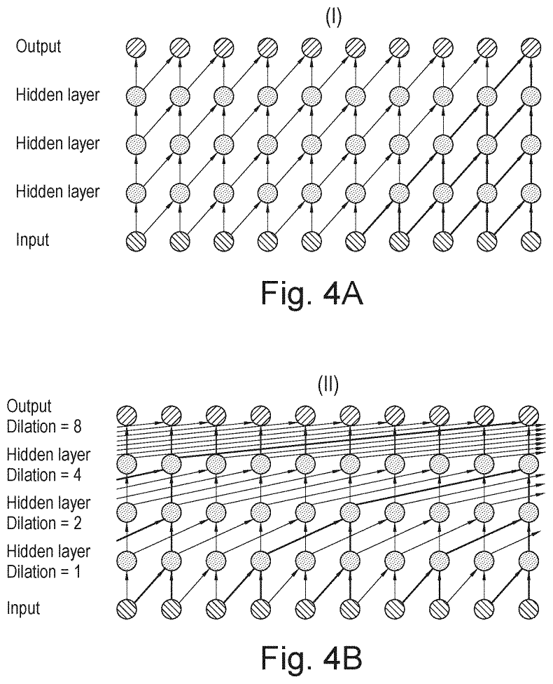

FIG. 4 illustrates the effects of operating a dilated neural network;

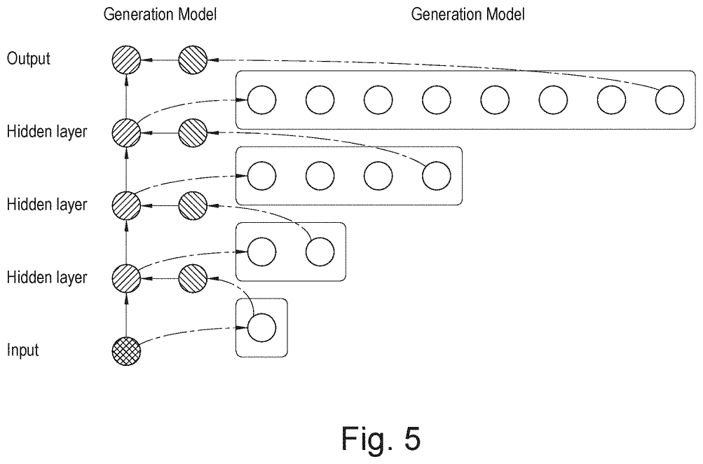

FIG. 5 illustrates a fast implementation to calculate the output of a dilated neural network for consecutive training datasets.

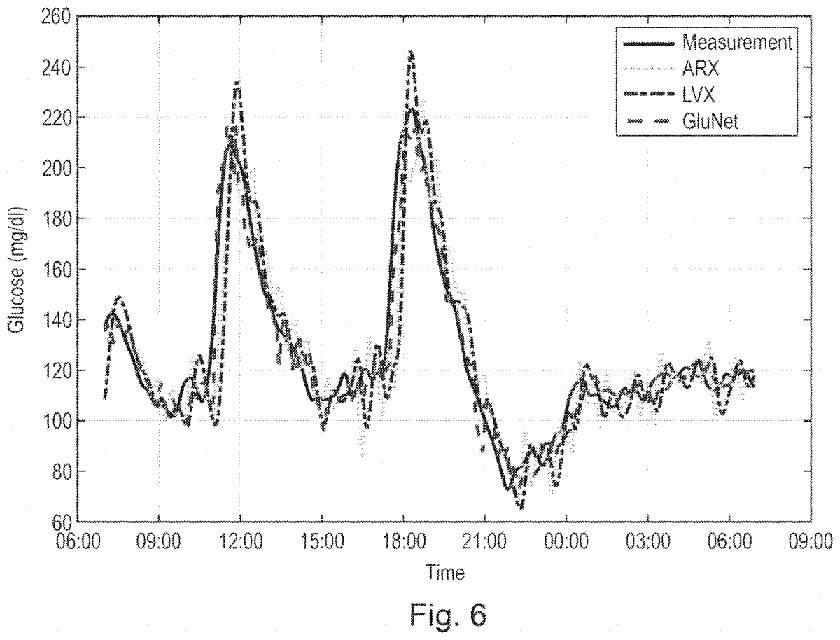

FIG. 6 is a graph demonstrating the prediction results given by an example embodiment for blood glucose prediction.

### DETAILED DESCRIPTION

Referring to the drawings, wherein like numbers denote like parts throughout the several views, FIG. 1 illustrates an example system capable of providing a prediction of the future value of a physiological parameter to the user.

The system disclosed in FIG. 1 comprises one or more data sources 100, one or more processors 120 and a display 140.

Each data source 100 may obtain or generate data relating to a particular parameter. For example, each data source 100 may comprise an input and/or a measurement element able to obtain data for use by the system 100. Although illustrated independently, one or more of the data sources 100 may be integrally formed with one another and/or any suitable combination of the processor 120 and display 140.

One of the data sources 100 is a physiological time-series data source 102 capable of obtaining time-series values (i.e. values associated with a time) of a physiological parameter which the system is to learn to predict. In the example shown, the physiological time-series data source 102 is a continuous glucose monitor. The skilled person will recognise that in other examples, alternative devices capable of measuring physiological parameters may be used. Alternatively, a user may manually enter data representing a physiological parameter which has been derived elsewhere, or data representing a physiological parameter may be generated by a patient simulator such as the UVA/Padova Type 1 Diabetes patient simulator.

There is also provided one or more further time-series data sources 104 capable of obtaining time-series values of one or more further parameters. For example, one of the further time-series data sources 104 may be a lifestyle time-series data source capable of obtaining time-series values describing lifestyle events of the patient such as meal intake, insulin injections and/or physical activity, or generating simulation data representing such lifestyle events. The further time-series data sources 104 may additionally or alternatively obtain time-series data regarding one or more further physiological parameters.

There may also be provided one or more time-invariant data sources 106 which are capable of obtaining a value for each of one or more time-invariant parameters. For example, one of the time-invariant data sources 106 may be a health attribute data source capable of obtaining a value for each of one or more health attributes of the patient such as age, weight and genetic factors, or generating simulation data representing such health attributes.

In some examples, one or more of the data sources wo may be wearable devices, such as smart watches or similar. Additionally or alternatively, data sources wo may comprise portable computing devices such as smartphones or tablets. In general, data sources wo may be any device capable of receiving an input relevant to the data or measuring data.

The processors 120 are able to carry out a computer-implemented method and receive and transmit data. In addition, the processors 120 implement a convolutional neural network (CNN) 300 which is described in greater detail with reference to FIG. 3.

Although only a single processor 120 is illustrated in FIG. 1, the skilled person will understand that functions of the processor may alternatively be implemented by a plurality of processing devices either co-located or at different locations. In such example, differing processing devices may be optimised for different tasks, such that implementation of the convolutional neural network, for example, may occur remote to the data sources 100.

The system of FIG. 1 further comprises a display 140 able to display information to the user and receive data.

The data sources 100 are connected to the computer system 120 by a communication channel, and the processors 120 to the display 140, such that the computer system 120 is able to receive data from the data sources wo and transmit data to the display 140. The data sources 100, processors 120 and display may be remote or co-located. For example, a single portable computing device, such as a mobile telephone, may act to receive multiple data inputs from a user (and thus act as multiple data sources), implement the CNN 300 (thus acting as the processors 120) and provide output on a display 140. This portable device may be in communication with a continuous glucose monitor (acting as the physiological time-series data source 102) over a short range communication channel such as Bluetooth. In some examples, the CNN 300 may be implemented remotely from the portable device, the device being in communication with the host of the CNN 300 via the Internet or similar.

Turning to FIG. 2, operation of the system 100 is now described. FIG. 2A describes a first, training, phase in the operation of the system, and FIG. 2B describes a second, prediction, phase.

Starting with FIG. 2A, at step 200, the data sources 100 obtain data, for example by measuring them using a sensor, or by simulating them. In particular, the physiological time-series data source 102 obtains time-series values of the physiological parameter, which in this example is the blood glucose level. The one or more further time-series data sources 104 obtain time-series values of the further parameters. In some examples comprising one or more time-invariant data sources 106, the one or more time-invariant data sources 106 may obtain a value for each of the time-invariant parameters.

At step 210, the data sources transmit all or part of the obtained data to the processors 120. The data received by the processors 120 at this stage is the training input.

At steps 221-224, the processors 120 pre-process the training input to generate one or more training examples.

At step 221, erroneous values in the training input, which could arise from sensor errors or mistakes in manually-inputted information at step 200, or transmission errors at step 210, may be screened and removed. Errors may for example be identified if, given basic knowledge about the patient (e.g. the patient is human), the parameter values are clearly wrong. For example, an error may be identified if the value (e.g. a concentration) is unrealistically large or small, or if there is an unusually large change over a short time. In this way, errors can be avoided which would arise in the prediction output of the method if much erroneous data remains.

At step 222, missing values in the training input, which could result from the removal of erroneous values or gaps in the data obtained by the data sources 100, may be completed, for example by interpolating them, by predicting them using a data-driven model or by inserting default values, or they may be left missing. Moreover, missing values may be selectively completed or left missing, for example based on whether a reasonable estimate can be given and/or whether the presence of a value is necessary at a future step of the method. For example, missing intervals of less than 1 hour may be completed, and missing intervals of more than 1 hour may be left missing. In this way, the lengths of the time intervals with no missing time-series values can be greatly increased, which allows generating many more training examples without compromising their quality.

At step 223, additional time-series values for further parameters not contained within the input are computed. These additional time-series values may be computed as a function of the cleaned and completed training input obtained by the end of step 222. In particular, one or more of these additional time-series values may represent modelled physiological parameters, and may be estimated using a physiological model based on the cleaned and completed training input. Usually, the modelled physiological parameters are chosen for their relevance to the physiological parameter that the method learns to predict, so that knowing the value of the modelled physiological parameters at one time provides information on the value and/or evolution of the physiological parameter which is to be predicted. For example, it might be known that there is a physical or biological interaction between the modelled physiological parameters and the physiological parameter that the method learns to predict. By computing the modelled physiological parameters, the knowledge encoded in the physiological model is incorporated into the input to the convolutional neural network. As a result, the input to the convolutional neural network is greatly enriched, so that training of the convolutional neural network is enhanced for a given training input. In particular, the parameters of the convolutional network do not need to independently account for the understood aspects of the physiological model used to generate additional parameters, since those assumptions and calculations are now provided in the input to the convolutional neural network. However, the use of a convolutional neural network may allow for adjustments and corrections of physiological models which provide imperfect results.

As an example, the physiological parameter which is to predicted may be blood glucose concentration, further parameters obtained from the data sources 100 may represent meal intake M (t) and insulin infusion I (t). Plasma insulin concentration Pi (t) and glucose rate of appearance Ra (t) may be additional parameters which are modelled from these values using a physiological mode. In one example, the relationship could be modelled using the following differential equations:

dS1/dt=I(t)−S1(t)/tmaxI

dS2/dt=(S1(t)−S2(t))/tmaxI

dPi/dt=−ke*Pi(t)+S2(t)/Vi/tmaxI

dRa1/dt=−Ra1(t)−Ag*M(t)/tmaxG

dRa/dt=−(Ra(t)−Ra1(t))/tmaxG

where S1, S2 and Ra1 are intermediate variables, and tmaxI, ke, Vi, Ag and tmaxG are empirically determined constants.

At step 224, training examples are generated. Each training example simulates what the task of predicting the physiological parameter would have been at a particular time in the past—the “training example time”—by associating a training dataset representing data that would have been available at the training example time (together with the values of any additional modelled parameters for that time) with a training label representing what the desired output of the prediction would have been. During training, the training dataset will be fed as input to the convolutional neural network and the training label as the target label.

Thus, each training dataset may be based on a sub-set of the data obtained by the end of step 223, where all the time-series values in the sub-set have a time-stamp preceding the training example time. In particular, all the time-series values in the sub-set may have their time-stamp comprised in a time window—the “training example time window”—that ends at the training example time. Further, the training datasets may be chosen to all have the same structure, so that the structure of the input to the convolutional neural network may be made invariant between training examples, thereby allowing for simpler convolutional neural network architecture. In particular, the dataset structure may be multiple time-series channels of a pre-determined length, where the time-series channels may be aligned (i.e. the time-stamps of each channel are synchronised) and/or regular (i.e. the interval between two consecutive time-stamps is constant within each channel and between channels). The dataset may also comprise zero or more time-invariant values.

For example, such a training dataset may be generated by: choosing a training example time window of sufficient length over which each of the time-series obtained by the end of step 223 has no missing values; transforming the time-series values of the physiological parameter to be regular if they are not already, for example by interpolation; and aligning the time-series values of the further parameters to those of the physiological parameter if they are not already, for example by interpolation.

The training examples may be defined for sequential training example times: for example, the training example time windows may all have the same predetermined length, but begin at sequential data points and end at sequential data points. In this way, a sliding window process is adopted across the training input to generate a large number of training examples for a given input across a time period greater than the length of the training example time windows.

The corresponding training label may be chosen to represent a value of the physiological parameter at a time after the training example time: the “training example prediction time” for that training example. In this case, the training example prediction time is the time for which the physiological parameter would have been predicted. In particular, the training example prediction time may be a predetermined prediction period after the training example time. Training the CNN to predict the physiological parameter a constant time period after the last time-series value in the training dataset, allows for a simple CNN architecture.

The training label may be the value of the physiological parameter at the training example prediction time, taken from the cleaned and completed training input obtained by the end of step 222. Alternatively, the training label may be the difference in value of the physiological parameter between the training example prediction time and the training example time. Such a differential encoding is advantageous where the physiological parameter is known to vary continuously, for example if it is known to be regulated by differential processes, since in that case the variation over a short time will be much smaller than the overall range of possible values for the physiological parameter. Furthermore, the variation of the physiological parameter over a short time may display some invariance with respect to the current value of the physiological parameter. As a result, the CNN may give more accurate predictions after training.

In addition to all of these variations, the training label may be quantised into one of a predetermined number of categories, such that the task of the convolutional neural network is to predict the correct category given the training dataset. This also allows simplifying the convolutional neural network architecture through the use of a categorical output layer. For example, the quantisation may be performed using a mu-law analogue-to-digital converter, which quantises a value x according to the formulae:

F(x)=sign(x)*ln(1+255|x|)/ln (256)

quantised(x)=min {255, max{0, floor((F(x)+1)*128)}}

thus outputting an integer comprised between 0 and 255.

At step 230, the processors 140 train the convolutional neural network 300, using the training examples obtained at step 224. Training the CNN 300 means calculating values for the weights that improve performance based on training examples, and updating the weights with the calculated values. For example, performance may be measured as the cross-entropy loss function on training examples, and improved weights may be found using a stochastic gradient descent method.

At start of training, the weights of the CNN 300 may be initialised randomly according to a distribution, or may be initialised with values obtained through a pre-training stage. In particular, the pre-training stage may involve training an unsupervised model such as a deep belief network or stacked de-noising auto-encoder, on data containing information relevant to prediction of the physiological parameter. Additionally or alternatively, the pre-training stage may involve training a pre-training CNN with the same architecture as the CNN 300, on data of the same format as the training examples. In this way, the number of training examples required to learn to predict the physiological parameter can be greatly reduced. For example, the pre-training CNN may be trained on data sourced from a large database of simulation and/or patient data, and the CNN 300 initialised with the weights of the pre-training CNN. When initialising the weights of the CNN 300 with those of the trained pre-training CNN, the adaptivity of the training technique to new examples (e.g. the learning rate of a stochastic gradient descent method) may be set to a larger value than the adaptivity of the pre-training CNN, so that the CNN 300 may quickly adapt to the particularities of the patient under training with the training examples.

Having trained the convolutional neural network, the system is now ready for the prediction phase, which is now described with reference to FIG. 2B.

At step 250, the data sources obtain data, as in step 200. Step 250 may be performed identically to step 200. However, the gathered data will not necessarily be the same, since it will be expected that the data obtained at step 250 will correspond to a different time than that obtained at step 200.

At step 260, the data sources transmit all or part of the data obtained at step 250 to the computer system. The prediction input comprises the data received by the computer system, and may further comprise all or part of the data previously received by the computer system i.e. the training input. Additionally, all or part of the prediction input may be appended to the training input, enabling the convolutional neural network to be subsequently further trained with the application of steps 222-224, thereby further improving accuracy.

At steps 271-274, the computer system pre-processes the prediction input to generate a prediction dataset. The prediction dataset encompasses values of the same parameters as included in a given training dataset, and where these are parameters which are time-variable includes a set of such values for a time period equal to the length of a training example time window.

At step 271, erroneous values in the prediction input may be screened and removed, as in step 221. Step 271 may be performed identically to step 221.

At step 272, missing values in the prediction input may be completed, as in step 222. Step 272 may be performed identically to step 222.

At step 273, time-series values of features may be computed as a function of the cleaned and completed prediction input obtained by the end of step 272, as in step 223. Step 273 may be performed identically to step 223.

At step 274, a prediction dataset is generated based on the data obtained by the end of step 223. If the training datasets have been chosen to all have the same structure, the prediction dataset may be generated to have the same structure. Step 274 may be performed identically to generating a training dataset in step 224.

At step 280, the processors 120 use the trained convolutional neural network obtained at step 230, to predict a prediction label from the prediction dataset. The prediction label represents the value of the physiological parameter at a prediction time, which can then be used by the user to inform decisions concerning treatment of the patient from which data was gathered. The prediction label may also be used to control the automatic operation of a medical device, such as a device configured to inject a therapeutic substance in a patient. For example, if the blood glucose concentration is predicted to rise above a certain value, such that the patient will be in severe hyperglycaemia, an implanted insulin pump may be controlled to inject insulin to the patient.

The aforementioned steps 200-280 may be performed serially, where each step is performed after the preceding one has completed, or may be performed in parallel, where the steps are performed simultaneously, and each step processes the data already processed by the previous steps. Furthermore, since data received through the prediction input may be used to further train the CNN, steps 221-230 may periodically be executed during the operation of steps 250-280 in order to further improve the accuracy of the physiological parameter prediction.

FIG. 3 illustrates an appropriate architecture for carrying out the steps mentioned above. The architecture comprises the convolutional neural network 300, a pre-processing layer 400 and label transform and recover layer 500.

The pre-processing layer 400 may receive an input 401 comprising time series values of the physiological parameter, which in this case is blood glucose concentration G(t). Additionally, insulin data I (402) and meal information M (403) is received by the pre-processing layer 400.

A dataset may then be generated according to steps 221-224/271-274 described above. In the particular embodiment shown, pre-processing comprises steps 410 to 450 (P1 to P5). Step 410 (P1) operates to rule out outliers in G(t), I and M (i.e. step 221/271). At steps 420 (P2) and 430 (P3), missing parameters are provided as per steps 222/272. In particular, at step 420 (P2), G(t) is interpolated when the missing data gap is not large, while at step 430 (P3), missing data in I and M is estimated according to models.

At step 440 (P4) (as per step 223/273), additional parameters are calculated to be input to the CNN 300. These may include plasma insulin estimation Pi and glucose rate of appearance Ra. Such parameters may be modelled from the data (G(t), I, M) contained with the input.

At step 450 (P5) (equivalent to steps 224/274 above), all parameters may be aligned with the same timeline in order to generate a final training or prediction dataset for input to the CNN 300. Moreover, the aligned blood glucose time series G′(t) is sent to the label transform 510, and the quantized change of blood glucose over a predetermined period after the end of a given dataset, ΔG′(t)=quantized(G(t+w)−G(t)) where w is the prediction period, is calculated and used as the label for training.

The convolutional neural network (CNN) 300 is a parametrised mathematical function which takes in an array 301 as input, which in this case is the aligned time-series of parameters obtained from step 450, and outputs a prediction label 360 representing a prediction of the physiological parameter. The learnable parameters of the CNN 300 are denoted weights. The CNN 300 comprises two stages: a deep neural network (DNN) 305, and a postprocessing neural network 350.

The deep neural network 305 comprises an optional causal convolution step 306, followed by one or more convolutional layers 310.

At step 306, a causal time-domain convolution may be performed on the input 301 according to an array of weights. The output of the convolution is sent to the input 312 of the first of the one or more convolutional layers 310.

Each of the one or more convolutional layers 310 takes as input 312 an array with one dimension representing time; at step 314, a time-domain convolution is performed on the input 312 according to an array of filter weights, to yield a convolution result array 316 with one dimension representing time. The convolution at step 314 may be causal and/or dilated. At step 318, an activation function is applied to yield an activations array 320. In the present embodiment, the activation function of step 318 is a hyperbolic tangent function, but could alternatively be replaced by a logistic function, a soft-max function, or a rectified linear unit (ReLU), among other possibilities.

The convolution of step 314 is said to be causal if every element of the convolution result array only depends on the elements of the input array that precede it in time.

The convolution of step 314 is said to be dilated with dilation D if values in the input array are skipped with step D in the time dimension. That is, for each element in the convolution result array, only certain values of the input array which are D steps apart in the time-dimension contribute to it. A dilated convolution with dilation 1 is identical to a non-dilated convolution. A dilated convolution has the following effect: in a CNN, each element in the output of a convolutional layer depends on a local subset of the input to that layer. The extent of the region in the CNN's input on which an element in the output of a layer depends on is said to be its receptive field. For a dilated convolutional layer with dilation D, the receptive fields of the elements in its output are increased by a factor of D, without requiring a greater number of weights. Information which originates from time-series values distant in time can thus be combined using fewer layers, thereby improving accuracy and reducing complexity. For example, in FIG. 4A, a non-dilated time-domain CNN with n=4 layers is illustrated. The bottom layer of nodes represents an input time-series with one channel, where each node is a time-series value in the channel. Each horizontal layer of nodes above represents the output time-series of each of the 4 convolutional layers. The receptive field of each element at the output of the top convolutional layer is found to be n+1=5. In contrast, in FIG. 4B, a dilated time-domain CNN with n=₄ layers is illustrated, where the i-th layer has dilation 2\hat{ }(i−1). The receptive field of each element at the output of the top convolutional layer can be seen to be 2\hat{ }n=16.

The CNN is said to be a causal CNN if at least one of the convolutional layers performs a causal convolution, and said to be a dilated CNN if at least one of the convolutional layers performs a dilated convolution with dilation greater than 1.

In addition, one or more of the convolutional layers 30 may feature a gated activation function, comprising the following steps. At step 322 a gate convolution may be performed according to an array of gate weights to yield a gate convolution result array 324. At step 328, a gate activation function may be applied to yield a gate activations array 330. The activations array 320, which was obtained at the end of step 318, is then multiplied element-wise with the gate activations array 330 to yield a gated activations array 332. Typically, the gate activation function 328 is chosen to take values between 0 and 1, such as a logistic function, so that each element in the gate activations array 330 can be thought of as a degree of confidence in the accuracy or significance of the corresponding element in the activations array 320. Such a feature increases the flexibility of the network, improving the overall accuracy of prediction.

Optionally, if a time-invariant data source 106 is present, one or more of the convolutional layers 310 may additionally take one or more of the time-invariant parameters as an additional input (not shown in FIG. 3). Linear combinations of the time-invariant parameters may be performed according to an array of weights and the result added element-wise to the convolution result array 316; additionally or alternatively, further linear combinations of the time-invariant parameters may be performed according to an array of weights and the result added element-wise to the gate convolution result array 324. This feature enables the CNN to take the time-invariant parameters into account in its prediction, and therefore be able to learn the differences in response of different patients that are correlated to the time-invariant parameters.

At step 334, the array of gated activations 332, or in the absence of a gated activation function, the array of activations 320, are multiplied by a matrix of weights, yielding an output 336.

At step 337, the output array 336 of the matrix multiplication may be added element-wise to the layer's input array 312, yielding an array of residuals 338. This feature, called a residual connection, improves the speed of training.

If the convolutional layer is followed by another convolutional layer 310, the array of residuals 338 may be sent to the following layer as input 312. Alternatively, the following layer may take as input 312 either of the output of the matrix multiplication 336, the gated activations 332, and the activations 320.

The post-processing neural network 350 comprises one or more fully-connected output layers, which perform linear combinations of their input, and then apply an activation function. In the example embodiment, three output layers 351-358 are used. The first layer, at steps 351 and 352, adds element-wise the outputs 336 of one or more of the convolutional layers of the DNN, and applies an activation function such as a rectified linear unit. If the output of another layer than the bottom convolutional layer is utilised, the post-processing neural network is said to feature skip connections. The second layer, at steps 354 and 355, multiplies the output of step 352 by a matrix of weights and applies an activation function such as a rectified linear unit. The third layer, at steps 357 and 358, is an output layer which multiplies the output of step 355 by a matrix of weights and applies a softmax activation function. The output of step 358 is a probability distribution over labels, which represents the probability distribution of the predicted label given the dataset, and is chosen to be the output 360 of the CNN.

The distribution over predicted labels is then sent to the label recover 520, where the distribution of the future value of the physiological parameter is estimated given the inverse label transform G(t+w)=G(t)+ΔG(t) where ΔG(t) is the de-quantised predicted label ΔG′(t).

FIG. 5 illustrates the technique of fast dilations, which may be used to simplify the computation at steps 230 (training the CNN) and 280 (prediction) in computing the outputs of the dilated convolutional layers. The key idea is that when calculating the convolutional layer outputs for datasets with overlapping time intervals, some computations overlap, so that may be reused. Exactly which computations may be re-used in a particular example is illustrated in FIG. 4B, which demonstrates the calculation of the outputs of the convolutional layers for a particular dataset. The outputs of the convolutional layers are represented by the nodes at which bold arrows arrive. The bottom set of nodes represent the input time-series to the convolutional layers. Each bold arrow represents the dependence of an output of a convolutional layer on an output of the previous convolutional layer or on the input time-series. Assuming that the outputs of the convolutional layers have already been calculated for datasets shifted back in time (represented by the grey arrows), all the nodes at which bold arrows arrive will already have been computed, except for the right-most nodes, which represent the latest value of the output of each convolutional layer, which have never been computed. Therefore, only the right-most nodes need to be computed; the others may be cached, assuming that they have already been computed when the convolutional layer outputs had already been calculated for previous datasets.

Furthermore, FIG. 4B also demonstrates how far back in time the previously-computed convolutional layer outputs necessary to the calculation of the latest convolutional layer outputs, had been computed. For example, the latest (right-most) output of the convolutional layer with dilation 2 depends on the output of the layer with dilation 1 calculated on a dataset 2 samples back. Similarly, the right-most output of the convolutional layer with dilation 4 depends on the output of the layer with dilation 2 calculated on a dataset 4 samples back. Thus those previously-calculated outputs may be cached using first-in-first-out (FIFO) queues of varying length, where the queue for layer 1 has length 2\hat{ }l.

The method of computing the layer outputs for consecutive datasets using the FIFO caching scheme is described according to FIG. 5. At each new sample, two steps are performed, one after the other: a pop step and a push step. At the pop step, the rightmost value of each FIFO is popped off, and the values are combined with the latest input sample to obtain the outputs of the layers. At the push phase, the latest input sample and the layer outputs have been calculated are pushed on the FIFOs, according to the diagram.

Thus, for L dilated convolutional layers, where layer 1 has dilation 2\hat{ }(l−1), the number of convolutional layer outputs is proportional to 2\hat{ }L−1. Without caching, every one of them would need to be recomputed at each dataset, even if the dataset samples overlap. However, if only the right-most nodes need to be recomputed at each dataset, the number of computations needed is proportional to L. Therefore, caching reduces the computational complexity of calculating the convolutional layer outputs from O(2\hat{ }L) to O(L). This speed-up is applicable to both the training and prediction steps, and the greatly reduced complexity enables the method to be carried out on low-power, wearable devices.

FIG. 6 shows experimental results demonstrating, for an embodiment according to the above disclosure, good performance at predicting the physiological parameter.

In this embodiment, the UVA/Padova Type 1 Diabetes simulator, is used as the data-gathering device, to provide 360 days' worth of time-series data for a single virtual patient, with 288 blood glucose concentration (BGC, the physiological parameter) data points per day, 1-5 insulin injection entries per day, 3 meal intake entries per day, and one exercise entry per day.

Pre-processing is performed according to steps 221-224, where erroneous values are identified and removed if the BGC is unrealistically large or small, or if the change in the BGC is unrealistically large. Gaps in the data of less than 1 hour are interpolated using cubic spline interpolation.

Plasma insulin concentration and glucose rate of appearance are chosen to be modelled parameters, calculated according to the differential equations disclosed at the description of step 223.

The first 180 days of data are used for training, and the last 180 days for prediction.

All the data in the first 180 days is processed into training examples according to the example method disclosed at the description of step 224. The training datasets are chosen to feature 5 channels of time-series values, representing the BGC, insulin infusion, meal intake, plasma insulin concentration and glucose rate of appearance respectively. The training labels are the difference between the BGC 30 minutes after the training dataset and the BGC at the end of the training dataset, quantised using a mu-law analogue-to-digital converter.

The CNN used is a dilated causal CNN 300 as described according to FIG. 3. The CNN 300 comprises 5 dilated causal convolutional layers 310 of depth 32, where the dilations of the layers are respectively 2, 4, 8, 16 and 32, and 3 fully-connected layers. The dilated causal convolutional layers 310 all feature a tanh activation function 318, a gated activation with a sigmoid gate function 328, multiplication by an array of weights 334 and residual connections 337. The first fully-connected layer is connected using skip connections 351 to the outputs 336 of all the dilated causal convolutional layers 310. The first and second fully-connected layers feature a ReLU activation function; the third fully-connected layer outputs a probability distribution over the predicted label using a softmax activation function.

The CNN is trained using stochastic gradient descent to minimise the cross-entropy loss function on the training examples. During training and prediction, the fast dilations technique is used to reduce the computational complexity of the dilated convolutional layer calculations.

After training, the CNN is used to predict the BGC 30 minutes ahead during the last 180 days of data, and its accuracy evaluated and compared to other methods.

FIG. 6 illustrates a sample 30-minute-ahead-prediction of BGC over 24 hours, generated by the trained CNN of this embodiment (dashed line), based on measured glucose levels (solid line). The predicted BGC comes close to the actual BGC. For comparison purposes, FIG. 6 also shows in the same graph BGC predictions made by previous modelling techniques trained on the same data. The dotted line illustrates the results of an autoregressive exogenous (ARX) model (described in D. A. Finan, F. J. Doyle III, C. C. Palerm, W. C. Bevier, H. C. Zisser, L. Jovanovi\check{ }c, and D. E. Seborg, “Experimental evaluation of a recursive model identification technique for type 1 diabetes,” Journal of diabetes science and technology, vol. 3, no. 5, pp. 1192-1202, 2009). The dash-dotted line illustrates the results of a latent variable with exogenous input (LVX) model (described in C. Zhao, E. Dassau, L. Jovanovi\check{ }c, H. C. Zisser, F. J. Doyle III, and D. E. Seborg, “Predicting subcutaneous glucose concentration using a latent variable based statistical method for type 1 diabetes mellitus,” Journal of diabetes science and technology, vol. 6, no. 3, pp. 617-633, 2012).

It can be seen from FIG. 6 that the approach of the present disclosure is able to predict BGC more accurately. In particular, results show less lag, fewer oscillations and less overshoot in comparison to previous techniques. Additionally, the approach of the present disclosure is advantageous since it requires minimal parameter tuning, with the CNN acting independently to optimise predictions. Furthermore, the approach of the present disclosure provides can not only provide a core expected value of a physiological parameter (as illustrated in FIG. 6) but also a probabilistic distribution, allowing a richer level of detail and permitting more appropriate risk analysis and response behaviours.

1. A computer-implemented method, comprising:
receiving a training input comprising time-series values of a physiological parameter and time-series values of one or more further parameters;
generating one or more training examples from the training input, wherein each training example comprises a training dataset and a corresponding training label, wherein each training dataset is generated from the training input with time-series values restricted to a time interval, and wherein each corresponding training label represents the value of the physiological parameter a prediction period after the end of that time interval; and
training a neural network using the training examples to generate a prediction label from a prediction dataset, wherein the prediction dataset is generated from a prediction input comprising time-series values of the physiological parameter and time-series values of one or more further parameters, and the prediction label represents the value of the physiological parameter a prediction period after the latest time-series value in the prediction dataset.

2. A method according to claim 1, wherein generating each training dataset includes modelling time-series values of additional parameters based on the training input.

3. A method according to claim 2, wherein one or more of the additional parameters represent modelled physiological parameters, and wherein modelling the additional parameters includes using a physiological model to estimate, from the training input, time-series values of the modelled physiological parameters.

4. A method according to claim 1, wherein each training label is the quantized change in the physiological parameter in the prediction period from the end of the time interval.

5. A method according to claim 1, wherein the neural network is a convolutional neural network.

6. A method according to claim 5, wherein the convolutional neural network is a causal convolutional neural network.

7. A method according to claim 5, wherein the convolutional neural network is a dilated convolutional neural network.

8. A method according to claim 1, wherein at least one output layer of the neural network is directly connected using skip connections to another layer.

9. A method according to claim 1, wherein the training input further comprises a value for each of one or more time-invariant parameters, and wherein the prediction input further comprises a value for each of the one or more time invariant parameters.

10. A method according to claim 9, wherein the neural network comprises at least one layer that operates as a function of one or more of the one or more time-invariant parameters.

11. A method according to claim 9, wherein one or more of the one or more time-invariant parameters represent factors varying between individuals which influence the evolution of the physiological parameter.

12. A method according to claim 1, wherein one or more of the neural network's layers includes a gated activation function.

13. A method according to claim 1, wherein generating the training examples includes removing outlier values and interpolating missing values in the time-series values of the training input.

14. A method according to claim 1, wherein one or more of the further parameters in the training input represent the occurrence of lifestyle events.

15. A method consisting in generating a prediction label representing a prediction of the physiological parameter from a prediction input, using the neural network trained by a method according to claim 1.

16. A method according to claim 15, further comprising using the prediction label to control the automatic operation of a device configured to inject a therapeutic substance in a patient.

17. A data processing system comprising a processor adapted to perform a method including:
receiving a training input comprising time-series values of a physiological parameter and time-series values of one or more further parameters;
generating one or more training examples from the training input, wherein each training example comprises a training dataset and a corresponding training label, wherein each training dataset is generated from the training input with time-series values restricted to a time interval, and wherein each corresponding training label represents the value of the physiological parameter a prediction period after the end of that time interval; and
training a neural network using the training examples to generate a prediction label from a prediction dataset, wherein the prediction dataset is generated from a prediction input comprising time-series values of the physiological parameter and time-series values of one or more further parameters, and the prediction label represents the value of the physiological parameter a prediction period after the latest time-series value in the prediction dataset.

18. A computer program comprising instructions which, when the program is executed by a computer, cause the computer to carry out a method including:
receiving a training input comprising time-series values of a physiological parameter and time-series values of one or more further parameters;
generating one or more training examples from the training input, wherein each training example comprises a training dataset and a corresponding training label, wherein each training dataset is generated from the training input with time-series values restricted to a time interval, and wherein each corresponding training label represents the value of the physiological parameter a prediction period after the end of that time interval; and
training a neural network using the training examples to generate a prediction label from a prediction dataset, wherein the prediction dataset is generated from a prediction input comprising time-series values of the physiological parameter and time-series values of one or more further parameters, and the prediction label represents the value of the physiological parameter a prediction period after the latest time-series value in the prediction dataset.

19. (canceled)
